(19) United States
(12) Patent Application Publication
(10) Pub. No.: US 2023/0217964 A1
(43) Pub. Date: Jul. 13, 2023

(54) Protein Beverage Composition

(71) Applicant: Cortez J. Hollis, Edina, MN (US)

(72) Inventor: Cortez J. Hollis, Edina, MN (US)

(21) Appl. No.: 17/571,917

(22) Filed: Jan. 10, 2022

(51) Int. Cl. A23L 2/66; A23L 2/68; A23L 2/60; A23L 2/56; A23L 33/19; A23L 33/155; A23L 33/175; A23L 33/16; A61K 31/4188; A61K 31/194; A61K 33/00; A61K 33/06; A61K 9/00

(52) U.S. Cl. CPC A23L 2/66; A23L 2/68; A23L 2/60; A23L 2/56; A23L 33/19; A23L 33/155; A23L 33/175; A23L 33/16; A61K 31/4188; A61K 31/194; A61K 33/00; A61K 33/06; A61K 9/0095; A23V 2002/00

(57) ABSTRACT

The present disclosure provides a protein beverage composition for a sports drink that refreshing and hydrating whilst also providing the nutrients required to assist with muscle recovery and growth after exercise in the form of a whey protein isolate, as well as containing a nutrient mix specifically designed to combat the adverse effects of alcohol consumption on recovery. The nutrient mix contains iron in a from such as ferrous citrate, biotin vitamin B7, and a mix of electrolytes for keeping the drinker hydrated and nourished when used as a mixer or chaser for an alcoholic beverage. The composition can be provided in any number of flavours.

### FIELD OF INVENTION

The present invention relates generally to a high protein sports beverage composition.

### BACKGROUND

In recent times the benefits of exercising and acquiring adequate nutrition for good recovery have become more and more recognized, and the fitness industry has thus grown exponentially. This is true also for the sports beverage industry.

There has been extensive research conducted on the role hydration plays in improving exercise performance, and how consumption of a hydrating protein composition can improve recovery and gains. Whey protein in particular has been identified as a highly effective ingredient.

Whey protein is a rich source of branched chain amino acids (BCAAs), containing the highest known levels of any natural food source. BCAAs are important for athletes, since, unlike the other essential amino acids, they are metabolized directly into muscle tissue and are the first amino acids used during periods of exercise and resistance training. Leucine may be important for athletes as it may play a key role in muscle protein synthesis and lean muscle support and growth. Research suggests that individuals who exercise benefit from diets high in leucine and may have more lean muscle tissue and less body fat than individuals whose diet contains lower levels of leucine. Whey protein isolate may have approximately 45% by weight more leucine than soy protein isolate.

While many current commercially available beverages containing whey protein exist, most of the available beverages only provide one of the aspects mentioned above, i.e., thirst quenching or delivery of nutrients. In particular, many beverages that offer nutritional benefits, e.g., protein shakes, but do not provide a consumer with a sense of refreshment, i.e., they do not quench a user's thirst.

A further consideration is that many individuals who are training and consume protein beverages also regularly consume alcohol.

Consumption of any amount of alcohol has been associated with a 40% reduction in the risk of iron deficiency anemia. Consumption of up to two alcoholic drinks per day seems to be associated with reduced risk of iron deficiency and iron deficiency anemia without a concomitant increase in the risk of iron overload.

Alcohol use in humans is also associated with a significant reduction in plasma biotin levels, and animal studies have shown inhibition in intestinal biotin absorption by chronic alcohol feeding. Alcohol is also a diuretic. It causes your body to remove fluids from your blood through your renal system, which includes the kidneys, ureters, and bladder, at a much quicker rate than other liquids. If you don't drink enough water with alcohol, you can become dehydrated quickly.

There remains a need in the art for a sports drink can be ingested in combination with alcohol and combat the adverse effects that alcohol has on recovery, while at the same time providing a beverage with a pleasing taste profile for consumer acceptance.

It is within this context that the present invention is provided.

### SUMMARY

The present disclosure provides a protein beverage composition for a sports drink that refreshing and hydrating whilst also providing the nutrients required to assist with muscle recovery and growth after exercise in the form of a whey protein isolate, as well as containing a nutrient mix specifically designed to combat the adverse effects of alcohol consumption on recovery. The nutrient mix contains iron in a from such as ferrous citrate, biotin vitamin B7, and a mix of electrolytes for keeping the drinker hydrated and nourished when used as a mixer or chaser for an alcoholic beverage. The composition can be provided in any number of flavours.

Thus, according to one aspect of the present disclosure there is provided a high protein, flavoured sports beverage composition, comprising: an amount of filtered or purified water in an amount between 85-97% relative to the weight of the beverage; an amount of whey protein isolate of at least 2% relative to the weight of the beverage, the whey protein isolate containing sunflower lechtin and one or more branched chain amino acids, BCAAs; one or more food acids; one or more sweeteners; one or more natural flavorings.

And wherein the composition further comprises a nutrient mix for combatting the adverse effects of alcohol on the body, the nutrient mix containing: biotin vitamin B7, Ferrous Citrate, one or more electrolyte compositions including one or more of sodium, potassium, magnesium, and calcium; and wherein the high protein, flavoured sports beverage composition is suitable for use as a mixer or chaser for an alcoholic beverage.

In some embodiments, the nutrient mix further comprises absorbic acid, vitamin C.

In some embodiments, the nutrient mix further comprises Niacinamide, Vitamin B-3.

In some embodiments the nutrient mix further comprises DL-Alpha Tocopheral Acetate, Vitamin E.

In some embodiments, the nutrient mix further comprises Retyinyl Palmitate, Vitamin A.

In some embodiments, the sweetener comprises a no calorie sweetener such as sucralose. In further embodiments, the sweetener further comprises Acesulfame potassium.

In some embodiments, the composition is designed and packaged such that a weight of 12 g or more of whey protein isolate is present in a serving.

In some embodiments, the BCAAs in the whey protein isolate include one or more of L-Isoleucine, L-Leucine, L-Valine.

In some embodiments, the one or more natural flavourings are selected to produce one of the following flavors: Blue raspberry, fruit punch, tropical, Chips, cookies, ice cream, and cake.

In some embodiments, the one or more food acids comprise Citric acid.

In some embodiments, the one or more food acids comprise Phosphoric acid.

In some embodiments, composition further comprises sodium citrate as an alkalinizing agent.

In some embodiments, composition further comprises Tri-potassium citrate as an emulsifying agent.

### BRIEF DESCRIPTION OF THE DRAWINGS

Various embodiments of the invention are disclosed in the following detailed description and accompanying drawings.

FIG. 1 illustrates an example table of components of a first flavour of a protein beverage composition according to the present disclosure.

FIG. 2 illustrates an example table of components of a second flavour of a protein beverage composition according to the present disclosure.

FIG. 3 illustrates an example table of components of a third flavour of a protein beverage composition according to the present disclosure.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present disclosure provides a protein beverage composition that is high in water and whey protein content, and which additionally comprises a nutrient mix containing Biotin, Iron, and electrolytes for combatting the adverse effects of alcohol consumption on recovery when the composition is used as a mixer or chaser in an alcoholic beverage.

As mentioned in the background section, alcohol is a diuretic and is known to deplete the body of biotin and electrolytes, dehydrating the user, and also interferes with iron level regulation.

There are currently no available protein drinks specifically designed to combat the adverse effects of alcohol consumption, despite the fact that many Whey protein isolates contain branched-chain amino acids (BCAAs) which are known to provide the body with amino acids (specifically the glucogenic amino acids alanine and glutamine) that assist liver function and help it to break down alcohol more efficiently so that a person can recover faster from a hangover.

The inventor of the present application has recognised the synergies between protein drink compositions and hangover recovery, and has developed a composition with increased iron content to combat iron regulation issues, and which contains biotin Vitamin B7, a vitamin not included in any other available sports drinks. Biotin, which as mentioned above is depleted by alcohol consumption, helps maintain many of the body's major systems, helping the body use enzymes and carrying nutrients throughout the body.

The beverage composition of the present disclosure is also high in water content and electrolytes—such as sodium, potassium, magnesium, and calcium—which help regulate fluid levels in the body after drinking alcohol, or while alcohol is being consumed.

The composition also comprises all natural flavours, and low calorie/zero sugar sweeteners, which combine to make a healthy refreshing protein drink that can easily accompany any alcohol as a chaser or mixer, killing the sharpness and bite in alcoholic drinks such as spirits and making them easier to swallow.

A preferred embodiment of the composition will now be described more specifically, followed by three example exact compositions with reference to FIGS. 1-3.

In the preferred embodiment, the composition is comprised mostly of filtered or purified water.

The second largest ingredient by weight is Whey protein isolate, the benefits of which for sports performance and recovery are well known in the art. Whey protein isolate (often referred to as “whey isolate” or “whey protein”) is a dietary supplement and food ingredient created by separating components from milk. Whey protein isolates are defined as having >90% protein on a dry weight basis

It is a complete, high-quality protein that contains all of the essential amino acids. In addition, it is very digestible, absorbed from your gut quickly compared with other types of protein. These qualities make it one of the best dietary sources of protein available.

As mentioned above, Whey protein isolate is also extremely abundant in branched-chain amino acids (BCAAs), which consist of the following three essential amino acids:

L-Isoleucine. Isoleucine is an α-amino acid that is used in the biosynthesis of proteins. Isoleucine, as one of the branched chain amino acids, is also critical in physiological functions of the whole body, such as growth, immunity, protein metabolism, fatty acid metabolism and glucose transportation. Isoleucine can improve the immune system, including immune organs, cells and reactive substances.

L-Leucine is an essential amino acid that is used in the biosynthesis of proteins. Leucine may help in healing skin and bones. It may increase muscle growth and lean body mass. It may increase production of human growth hormone (HGH). It may help control blood sugar.

L-Valine. Valine is an α-amino acid that is used in the biosynthesis of proteins It is essential in humans, meaning the body cannot synthesize it: it must be obtained from the diet. L-valine is a branched-chain essential amino acid (BCAA) that has stimulant activity. It promotes muscle growth and tissue repair. It is a precursor in the penicillin biosynthetic pathway.

Preferably, the Whey isolate also contains sunflower lecithin, which comprises a collection of phospholipids that make it a natural emulsifier and has been proven to have various secondary benefits including strengthening the nervous system, reducing stress levels and lowering blood pressure, improving liver, cardiovascular, and bone and joint health, acting as an anti-oxidant, and improving digestion and wound recovery.

As mentioned above, consumers are looking for a convenient manner to refresh themselves and ingest nutrients. Many consumers lack certain vitamins in their diet and thus look to supplements to obtain such nutrients. The composition thus also comprises a specially formulated nutrient mix for combatting the adverse effects of alcohol consumption. A crucial ingredient of the nutrient mix is Vitamin B7, or Biotin, which is known to be depleted during alcohol consumption.

One of biotin's primary functions is its ability to metabolize carbohydrates, proteins, and fats. This vitamin also functions as a coenzyme and aids in synthesizing fatty acids and certain amino acids. Without the B complex vitamins, the body cannot use the energy that from food consumed.

Biotin is also one of the vitamins responsible for rebuilding muscle strength and helping tissues grow. Neither muscle nor tissue lasts forever, and the body relies on B vitamins to help rebuild after tissues break down or muscles sustain damage. This vitamin also has a key role in alleviating muscle and joint inflammation, aches, and pains.

Along with the other B complex vitamins, biotin keeps the nervous system in working order by assisting with neurotransmitter activity and helping with nerve signals. Together, B vitamins also protect the brain, improve the memory, and help form a defense against cognitive issues and neurodegenerative disorders. Along with preventing dementia and Alzheimer's disease, biotin and the other B vitamins can help you better the concentration and keep a positive outlook.

The nutrient is also essential for healthy hair, skin, and nails—biotin supplements are well known as a way of causing hair to thicken, grow faster, and become more lustrous.

Another crucial ingredient of the nutrient mix is iron, another chemical known to be depleted in the body by alcohol consumption.

Iron may be provided in the form of Ferrous Citrate, which helps maintain normal blood iron levels and prevents the body from experiencing iron deficiency, as well as the range of mild to severe side effects that often accompany it. Ferrous citrate is commonly used to treat iron deficiency anemia.

The nutrient mix can also contain a number of other vitamins for assisting with recovery and hydration, including but not limited to: Ascorbic Acid (Vitamin C), a potent reducing and antioxidant agent that functions in fighting bacterial infections, in detoxifying reactions, and in the formation of collagen in fibrous tissue, teeth, bones, connective tissue, skin, and capillaries; Niacinamide (Vitamin B-3), a form of vitamin B3 (niacin) that plays an important role in energy metabolism and cell health, DL-Alpha Tocopheral Acetate (Vitamin E) which can aid the composition in achieving a longer shelf life; and Retyinyl Palmitate (Vitamin A), which supports and maintains optimum eye health, immune system health, and reproductive health.

The nutrient mix will also contain a number of electrolytes and dietary minerals, such as, for example, calcium, Chloride, Chromium, Cobalt (as part of Vitamin B12), Copper, Iodine, Iron, Magnesium, Manganese, Molybdenum, Phosphorus, Potassium, Selenium, Sodium, and Zinc. Additionally, it is also contemplated that the protein-containing beverage includes one or more amino acids such as Isoleucine, Lysine, Leucine, Methionine, Phenylalanine, Threonine, Tryptophan, Valine and Histidine.

The composition will generally include Citric Acid. Citric acid is a weak organic acid with the chemical formula HOC(CH₂CO₂H)₂. It is an intermediate in the citric acid cycle, which occurs in the metabolism of all aerobic organisms, and thus helps keep the protein composition fresher longer.

The composition will generally include Phosphoric Acid, which works with the body to form strong bones and teeth and aids the muscles recover after exercise.

The composition also contains zero calorie naturally occurring sweeteners to provide a refreshing and enjoyable taste to the consumer.

A preferred sweetener is Sucralose (brand name Splenda). Sucralose is a no-calorie sweetener that can be used to lower one's intake of added sugars. It is about 600 times sweeter than sugar, so only small amounts of sucralose are used to match the sweetness provided by sugar. By making a simple swap of sucralose for sugar, it can help you limit calorie intake. This can be especially helpful for people with diabetes who need to monitor their sugar intake.

Another preferred sweetener is Acesulfame Potassium. Acesulfame potassium is often used in sweetener blends to produce a more sugar-like taste in a food or beverage. Since acesulfame K is not metabolized, it also contributes no calories.

Other ingredients of the composition can include: Sodium Citrate, which is used as alkalinizing agent, and Tri-potassium citrate. Which is used as a buffering, sequestering or emulsifying agent—it usually replaces trisodium citrate whenever a low sodium content is desired.

Having described a general formulation of the protein beverage composition of the present disclosure, specific example compositions will now be listed with reference to the figures.

Referring to FIG. 1, an example table of components of a first flavour (Blue Raspberry) of a protein beverage composition according to the present disclosure is shown.

The nutrient mix described above is listed as a “protein water premix”. The exact ratio of the ingredients of the nutrients listed can vary between different premixes.

The ingredients list is as follows: Filtered Water, Whey Protein Isolate (Contains Sunflower Lecithin), Natural Flavors, Citric Acid, Phosphoric Acid, Sucralose, Acesulfame Potassium, Sodium Citrate, Tri Potassium Citrate, L-Isoleucine, L-Leucine, L-Valine, Ascorbic Acid (Vitamin C), Niacinamide (Vitamin B-3), dl-Alpha Tocopheral Acetate, Ferrous Citrate, Retyinyl Palmitate (Vitamin A), FD&C Blue 1, Biotin

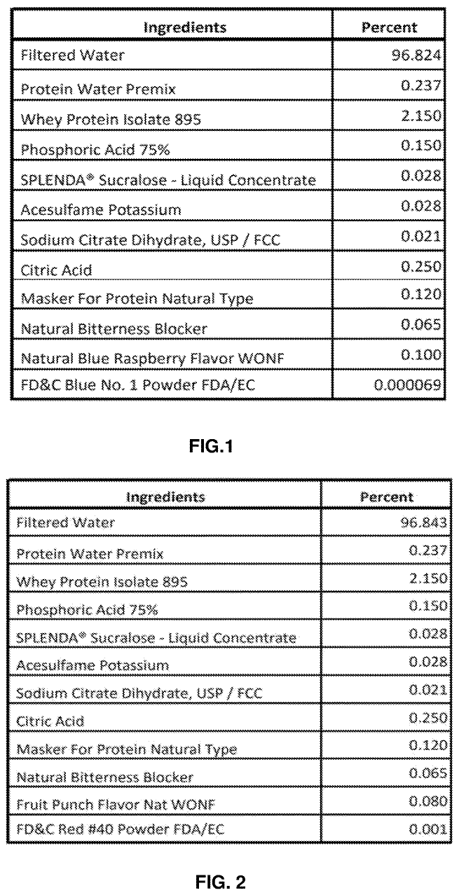

Referring to FIG. 2, an example table of components of a first flavour (Fruit Punch) of a protein beverage composition according to the present disclosure is shown.

The nutrient mix described above is listed as a “protein water premix”. The exact ratio of the ingredients of the nutrients listed can vary between different premixes.

The ingredients list is as follows: Filtered Water, Whey Protein Isolate (Contains Sunflower Lecithin), Natural Flavors, Citric Acid, Phosphoric Acid, Sucralose, Acesulfame Potassium, Sodium Citrate, Tri Potassium Citrate, L-Isoleucine, L-Leucine, L-Valine, Ascorbic Acid (Vitamin C), Niacinamide (Vitamin B-3), FD&C Red 40, dl-Alpha Tocopheral Acetate, Ferrous Citrate, Retyinyl Palmitate (Vitamin A), Biotin

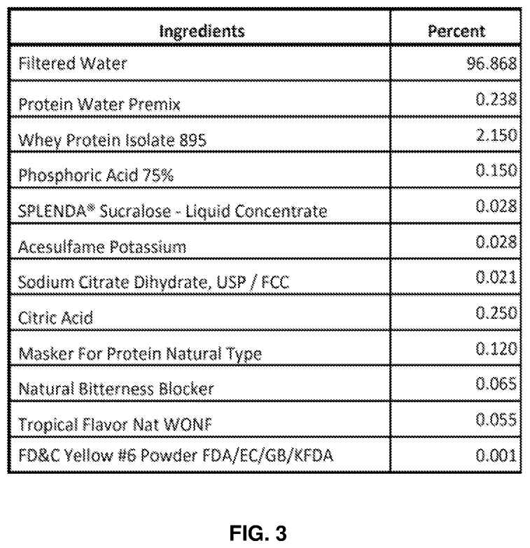

Referring to FIG. 3, an example table of components of a first flavour (Tropical) of a protein beverage composition according to the present disclosure is shown.

The nutrient mix described above is listed as a “protein water premix”. The exact ratio of the ingredients of the nutrients listed can vary between different premixes.

The ingredients list is as follows: Filtered Water, Whey Protein Isolate (Contains Sunflower Lecithin), Citric Acid, Natural Flavors, Phosphoric Acid, Sucralose, Acesulfame Potassium, Sodium Citrate, Tri Potassium Citrate, L-Isoleucine, L-Leucine, L-Valine, Ascorbic Acid (Vitamin C), Niacinamide (Vitamin B-3), dl-Alpha Tocopheral Acetate, Ferrous Citrate, FD&C Yellow 6, Retyinyl Palmitate (Vitamin A), Biotin

As can be seen, the formula of the different flavoured compositions is generally similar in structure.

When consumed as a mixer or chaser in an alcoholic beverage, the ratio of the protein composition to alcoholic beverage should be at least 2:1 or greater.

It is contemplated that the components of the protein-containing beverage may be combined in any manner and in any order. In one embodiment, the protein-containing beverage is made in a master batch and then divided into smaller portions.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the protein composition have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A high protein, flavoured sports beverage composition, comprising:
an amount of filtered or purified water in an amount between 85-97% relative to the weight of the beverage;
an amount of whey protein isolate of at least 2% relative to the weight of the beverage, the whey protein isolate containing sunflower lechtin and one or more branched chain amino acids, BCAAs;
one or more food acids;
one or more sweeteners;
one or more natural flavorings; and
a nutrient mix for combatting the adverse effects of alcohol on the body, the nutrient mix containing: biotin vitamin B7, Ferrous Citrate, one or more electrolyte compositions including one or more of sodium, potassium, magnesium, and calcium;
wherein the high protein, flavoured sports beverage composition is suitable for use as a mixer or chaser for an alcoholic beverage.

2. A high protein, flavoured beverage composition according to claim 1, wherein the nutrient mix further comprises absorbic acid, vitamin C.

3. A high protein, flavoured beverage composition according to claim 1, wherein the nutrient mix further comprises Niacinamide, Vitamin B-3.

4. A high protein, flavoured beverage composition according to claim 1, wherein the nutrient mix further comprises DL-Alpha Tocopheral Acetate, Vitamin E.

5. A high protein, flavoured beverage composition according to claim 1, wherein the nutrient mix further comprises Retyinyl Palmitate, Vitamin A.

6. A high protein, flavoured beverage composition according to claim 1, wherein the sweetener comprises a no calorie sweetener such as sucralose.

7. A high protein, flavoured beverage composition according to claim 6, wherein the sweetener further comprises Acesulfame potassium.

8. A high protein, flavoured beverage composition according to claim 1, wherein the composition is designed and packaged such that a weight of 12 g or more of whey protein isolate is present in a serving.

9. A high protein, flavoured beverage composition according to claim 1, wherein the BCAAs in the whey protein isolate include one or more of L-Isoleucine, L-Leucine, L-Valine.

10. A high protein, flavoured beverage composition according to claim 1, wherein the one or more natural flavourings are selected to produce one of the following flavors: Blue raspberry, fruit punch, tropical, Chips, cookies, ice cream, and cake.

11. A high protein, flavoured beverage composition according to claim 1, wherein the one or more food acids comprise Citric acid.

12. A high protein, flavoured beverage composition according to claim 1, wherein the one or more food acids comprise Phosphoric acid.

13. A high protein, flavoured beverage composition according to claim 1, wherein composition further comprises sodium citrate as an alkalinizing agent.

14. A high protein, flavoured beverage composition according to claim 1, wherein composition further comprises Tri-potassium citrate as an emulsifying agent.
